(19) United States
(12) Patent Application Publication
(10) Pub. No.: US 2019/0091131 A1
(43) Pub. Date: Mar. 28, 2019

(54) STRIPPABLE AQUEOUS COSMETIC NAIL VARNISH COMPOSITION CONTAINING STABILISED POLYURETHANE

(71) Applicant: FlABILA, MAINTENON (FR)

(72) Inventor: Serge MAGNET, BUC (FR)

(21) Appl. No.: 15/578,830

(22) PCT Filed: Jun. 2, 2016
(86) PCT No.: PCT/FR2016/051317

§ 371 Date: Dec. 1, 2017

(30) Foreign Application Priority Data
Jun. 3, 2015 (FR) 1555058

(51) Int. Cl. A61K 8/87; A61Q 3/02; A61K 8/25

(52) U.S. Cl. CPC A61K 8/87; A61K 2800/413; A61K 8/25; A61Q 3/02

(57) ABSTRACT

The invention relates to a strippable aqueous nail varnish composition containing an aqueous polyurethane dispersion as a film-forming agent, characterized in that it also comprises an aqueous anionic colloidal silica dispersion containing sodium or ammonium counter-ions, preferably containing silica particles measuring between 5 and 45 nm. The presence of said colloidal silica stabilizes the polyurethane dispersion, by preventing sedimentation, particularly when the varnish composition contains pearls or glitter dust.

The present invention relates to aqueous, colorless or colored nail varnishes, and more particularly varnishes that are removable by stripping.

### PRIOR ART

The nail varnish market is now dominated by the nail varnish compositions prepared in a solvent phase and are based on nitrocellulose combined with another resin and with plasticizers in solution in volatile solvents. Environmental considerations connected with the evaporation of these solvents in the atmosphere during application of these varnishes as well as the potential risks associated with the flammability of these solvents have led manufacturers to develop aqueous nail varnishes.

However, water-based nail varnishes give lower durability on the nail than organic solvent-based varnishes and are more difficult to remove. For this purpose, however, they require the same organic nail varnish removers as the nonaqueous-solvent-based nail varnishes.

Aqueous nail varnishes have been developed that can be removed by simple peeling (or stripping), an operation consisting of removing the deposited film from the nail by starting to strip it at a corner of the nail, lifting it and pulling it by this end to detach it from the nail, ideally in one piece. Peelable aqueous varnishes of this kind do not require the use of a nail varnish remover for makeup removal and thus represent the most ecological technical solution.

These peelable (or strippable) aqueous varnishes generally contain an aqueous polyurethane dispersion as the film-forming agent. Such dispersions are mentioned in numerous patents.

Thus, patent EP0143480 A2 describes a strippable aqueous varnish prepared from aqueous polyurethane dispersions formed from polyethers or polyesters with aliphatic or aromatic isocyanates.

Patent EP391322 B1 describes the use of an aqueous dispersion of a polyurethane binder and/or a polyurethane/acrylate copolymer in the nail varnish composition, in the presence of an acrylic thickener.

Moreover, patent FR2711059 claims the use of an anionic polyester-polyurethane aqueous dispersion containing particles with a size between 2 and 40 nm and giving a film with improved hardness.

Patent EP0423471 describes a nail varnish composition based on a polyurethane-polyurea aqueous dispersion. To improve durability on the nail, patent FR2718350 proposes a combination of an aqueous dispersion of polyurethane particles and radical polymers with carboxyl groups, in proportions that depend on the respective glass transition temperatures of the polymers.

Combinations of various polyurethane dispersions having different elongations at break are also described in patent U.S. Pat. No. 5,830,443 for forming a varnish that is hard, but flexible. Finally, patent FR2831797 describes an aqueous nail varnish based on an aqueous dispersion of a specific polyurethane, obtained by polycondensation of tetramethyl xylylene diisocyanate with a diol.

These patents also describe the use of the additives and other ingredients that are used in the composition of aqueous nail varnishes and are known to those skilled in the art.

However, technical limitations have been observed with respect to the preparation and use of these nail varnishes based on a polyurethane dispersion, notably difficulties with stabilizing dense mineral pigments such as metal oxides and fillers providing particular effects such as nacres. In fact these pigments and fillers tend to sediment over time despite the use of additives for controlling the rheology of the varnishes. This phenomenon proceeds more quickly the higher the temperature.

The associative acrylic and polyurethane thickeners generally used for adjusting the rheological profile of the aqueous preparations very often give the varnish an unsuitable structure, which is reflected in poor behavior during application as well as phase separations, called syneresis, in stability tests. The use of rheology agents based on cellulose or natural gums generally causes a loss of transparency of the preparation based on a polyurethane dispersion, which turns white; this is reflected during coloration of the varnish by a color difference between the liquid varnish and the dry film applied on the nail. The mineral antisedimentation agents such as bentonites and aluminum silicates display limited compatibility with the polyurethane dispersions and they are difficult to disperse in the medium. They also affect the gloss of the varnish film.

### AIMS OF THE INVENTION

A first aim of the present invention is to overcome the drawbacks of the peelable varnish compositions of the prior art and to propose a colorless or colored aqueous nail varnish composition prepared with an aqueous polyurethane dispersion as film-forming agent, which displays improved stability in storage, while maintaining the desired application properties and aesthetic effect, and that can be removed by simple stripping.

Another aim of the invention is to propose an aqueous nail varnish composition prepared with an aqueous polyurethane dispersion as film-forming agent, which gives a glossy film after application that is sufficiently durable and water-resistant and can be removed easily by simple stripping.

### DESCRIPTION OF THE INVENTION

For this purpose, the present invention proposes a strippable aqueous nail varnish composition, containing an aqueous polyurethane dispersion as film-forming agent, characterized in that it also comprises at least 1 wt % of an aqueous dispersion of anionic colloidal silica containing sodium or ammonium counter-ions and between 15 and 50 wt % of silica particles.

The inventors in fact discovered that the use of an aqueous dispersion of colloidal silica as an antisedimentation agent makes it possible, owing to the interactions between the chemical groups present on the surface of the dispersed silica particles and the surface of the polyurethane particles, and the surface of the pigments and fillers optionally present, to endow the whole of the varnish composition with improved stability. This stability is better than that supplied by the rheology additives used in the prior art for compositions of strippable aqueous varnish of this kind.

The advantage of the present invention is therefore that a nail varnish is obtained that does not display sedimentation, notably of the pigments and fillers in the bottles, while giving, after application, a glossy film that is sufficiently durable and water-resistant. This composition according to the invention is able to form a film on the nail, which after drying can easily be removed by simple stripping.

Advantageously, the particle size of the colloidal silica is between 5 and 45 nm, preferably between 5 and 30 nm, and more preferably between 7 and 25 nm. The size distribution of these particles, which are therefore nanoparticles, may be a mono- or poly-dispersed distribution.

The aqueous dispersion of colloidal silica preferably contains between 30 and 40 wt % of silica particles, i.e. a dry extract between 15 and 50 wt % and preferably between 30 and 40 wt %.

The antisedimentation effects of the dispersion of colloidal silica have been found to be particularly advantageous when the aqueous nail varnish composition comprises a proportion of colloidal silica dispersion between 1 and 10 wt %, preferably between 2 and 5 wt %, relative to the total weight of the composition. The minimum amount of colloidal silica to add depends in particular on the nature of the pigments and fillers included in the varnish formulation. For example, as nacre particles are larger and heavier than particles of organic pigments, they require a higher concentration of colloidal silica.

The present invention applies in particular to aqueous nail varnish compositions in which the polyurethane dispersion is an anionic dispersion, based on polyacrylate, polyether, polyester or polycarbonate and aliphatic and/or aromatic isocyanate, or a mixture thereof.

The dry extract of the polyurethane dispersion may be between 20 and 50 wt %, and preferably between 20 and 40 wt %. Nonlimiting examples of polyurethane dispersions are notably the dispersions Alberdingk U6800 or Alberdingk U5200 marketed by the company Alberdingk-Boley, or the dispersion Baycusan C1004 marketed by the company Bayer.

The polyurethane dispersion advantageously contains particles smaller than 200 nm, and preferably smaller than 100 nm, for preparing nail varnish compositions that are transparent or translucent before the addition of dyes or pigments.

Advantageously, the polyurethane dispersion has a temperature of film formation less than or equal to 30° C. and preferably less than 20° C. in order to ensure good film-forming properties and an elongation at break of 100% or more.

The nail varnish composition according to the present invention is prepared by simple mixing of the various constituents of the composition with the polyurethane dispersion, and then adding and mixing the aqueous dispersion of colloidal silica, for example using a dispersing machine.

The composition according to the present invention may contain between 80% and 98%, preferably between 85 and 97%, of aqueous polyurethane dispersion.

The composition according to the present invention may contain one or more organic or inorganic pigments and/or fillers (for example nacres) in a proportion less than or equal to 10 wt %, preferably less than or equal to 5 wt %, relative to the total weight of the composition.

The varnish composition according to the present invention may also contain the additives and ingredients commonly used for preparing aqueous nail varnish. In particular it may contain at least one additive selected from: co-solvents, coalescing agents, antifoaming agents, surfactants, wetting agents, dispersants, waxes, silicones, drying accelerators, crosslinking agents, adhesion promoters, UV filters, rheology agents such as associative acrylic and polyurethane thickeners, preservatives, such as antibacterial and antifungal agents necessary for preservation of the aqueous medium, or a mixture thereof.

The composition according to the present invention may be colored using the water-soluble dyes or the organic and inorganic pigments and lakes permitted by the legislation concerning cosmetics.

The composition according to the present invention may also contain one or more active agents for maintaining and improving the appearance of the nail, preferably with a nontherapeutic effect, for example agents that improve the smoothness of the nail or that provide a hydrating action.

The nail varnish composition according to the invention may also contain, as complementary film-forming agent (also called co-binder), preferably up to 30 wt % of an aqueous dispersion of acrylic or styrene acrylic polymer with particle size under 150 nm, and preferably under 100 nm, so as not to opacify the composition excessively. The content of acrylic or styrene acrylic copolymer in such a dispersion is preferably between 30 and 50%. If the temperature of film formation of the acrylic or styrene acrylic dispersion is above 20° C., it is lowered to a value less than or equal to 25° C. by adding a coalescing agent to the composition according to a technique that is widely known to those skilled in the art. A nonlimiting example of such an acrylic dispersion is Neocryl A1131 marketed by the company DSM.

The composition according to the invention is thus able to form a film on the nail which, after drying, can be removed by simple stripping.

The present invention also relates to a method for nail makeup and makeup removal characterized in that it comprises the following steps:

i) applying one or more layers of a varnish composition as described above, in the form of a continuous film, on the nail

ii) drying each of the layers of varnish at room temperature

iii) removing the varnish by peeling the film deposited on the nail.

Room temperature means a temperature between about 15 and 25° C.

The present invention will now be described in more detail and illustrated by the nonlimiting examples given below:

### EXAMPLES

Throughout the text, unless stated otherwise, the percentages are percentages by weight.

Various peelable nail varnish compositions were formulated and tested.

The general formula of the nail varnish composition, called basic composition, which is colorless, comprises the following constituents:

| Aqueous polyurethane dispersion (e.g. Alberdingk U5200) | 81 to 96 |
|---|---|
| Aqueous dispersion of antisedimentation agent | 15 |
| from 0 (comparative examples) to | |
| Cosolvent (Dowanol DPM): propylene glycol derivative | 2 |
| Bactericide (Euxyl 90/10) | 1 |
| Associative thickener of the HEUR type | 0.5 |
| Antifoaming agent | 0.25 |
| Surfactant | 0.25 |
| | 100 |

The following tests were performed on the various varnish compositions:
Gloss: 100 μm of the varnish composition is applied on a Leneta card. After drying the film obtained at 20° C., its gloss is measured at an angle of 60° using a Byk Gardner glossmeter.
   The gloss of the colored varnish is measured on the film obtained from the colorless varnish, to which 3% of pigment dispersion containing 30% of titanium oxide has been added.
Stability at 45° C.: A bottle is filled with nail varnish composition up to three quarters of its height, then stoppered and put in a stove at 45° C. for a month. No phenomenon of sedimentation, phase separation, demixing of color or caking is to be observed.
Stability at 20° C.: A bottle is filled with nail varnish composition up to three quarters of its height, then stoppered and stored at 20° C. for several months. No phenomenon of sedimentation, phase separation, demixing of color or caking is to be observed.
Dry extract: Between 0.5 g and 1 g of varnish composition is weighed in a dish. Then this stove plate is put in an electric desiccator at 140° C. connected to a balance until the weight is completely stable.
Peelability: the varnish composition is applied on the nail and then dried at room temperature. The film obtained is then removed from the nail by stripping; starting at a corner of the nail and pulling it. The test is positive if the varnish film is removed in one piece without breaking and without leaving bits on the nail.
Appearance of the film: A varnish composition is applied in the form of a film of 100 micrometers when wet, on a glass plate using an applicator, and then dried at 20° C. Absence of cracking, absence of grains and transparency of the film are observed.

The various compositions formulated and tested are presented in the following examples 1 to 16.

### Comparative Example 1 (Reference)

Colorless varnish according to the general formula, not containing antisedimentation agent.

### Example 2

Colorless varnish according to the present invention, prepared according to the general formula, prepared with 5% of colloidal silica dispersion with particle size of 22 nm in water, for example Ludox AS40 marketed by Grace or Bindzil 40NH3130 marketed by AKZO Nobel.

### Example 3 (Comparative)

Colorless varnish according to the general formula, prepared with 5% of 10% aqueous dispersion of synthetic silicate (Laponite XLS marketed by Rockwood).

### Example 4 (Comparative)

Colorless varnish according to the general formula, prepared with 15% of a 5% aqueous gel of bentonite (Optigel CK marketed by Rockwood).

### Example 5 (Comparative)

Colorless varnish according to the general formula, prepared with 15% of a 5% aqueous gel of associative thickener of type ASE: the acrylic thickener Acrysol ASE60 marketed by DOW Chemicals.

### Example 6 (Comparative)

Colorless varnish according to the general formula, prepared with 12% of a 5% aqueous gel of hydroxycellulose (Natrosol 250HR marketed by Ashland).

Results:

The characteristics of the colorless varnishes obtained in examples 1 to 6 are presented in Table 1 below:

TABLE 1

| | Ex 1 | | Ex 3 | Ex 4 | Ex 5 | Ex 6 |
|---|---|---|---|---|---|---|
| | (Ref.) | Ex 2 | (comp.) | (comp.) | (comp.) | (comp.) |
| Anti-sedimentation | / | colloidal | silicate | bentonite | acrylic | hydro- |
| agent | | silica | | | thickener | cellulose |
| | | | | | | gel |
| Appearance of the | translucent | translucent | translucent | turbid | opaque | opaque |
| base | | | | | | |
| Appearance of the | transparent | transparent | transparent | turbid | turbid | opaque |
| film | | | | | | |
| Dry extract (%) | 38 | 38.1 | 36.4 | 36.4 | 36.4 | 36.4 |
| Gloss (60°, UB) | 94 | 95 | 85 | 70 | 70 | 70 |
| Peeling | OK | OK | OK | OK | OK | OK |

Appearance of the base: refers to the visual appearance of the composition prepared, before application (in the bottle);

Appearance of the film: refers to the visual appearance of the varnish after application on a glass plate.

It appears that the dispersion of colloidal silica (example 2) has the best compatibility with the aqueous polyurethane dispersion, which is demonstrated by the transparency and gloss of the varnish film obtained.

### Example 7

Colorless varnish according to the present invention, prepared according to the general formula with 1% of aqueous dispersion of colloidal silica with particle size of 22 nm neutralized with ammonia, for example Ludox AS40 marketed by Grace. Identical results were obtained with Bindzil 40NH3130 marketed by AKZO Nobel.

### Example 8

Colorless varnish according to the present invention, prepared according to the general formula with 2% of aqueous dispersion of colloidal silica with particle size of 22 nm neutralized with ammonia, for example Ludox AS40 marketed by Grace. Identical results were obtained with Bindzil 40NH3130 marketed by AKZO Nobel.

### Example 9

Colorless varnish according to the present invention, prepared according to the general formula with 1% of 30% aqueous dispersion of colloidal silica with particle size of 17 nm neutralized with ammonia: Levasil 200N30 marketed by AKZO Nobel.

### Example 10

Colorless varnish according to the present invention, prepared according to the general formula with 2% of 30% aqueous dispersion of colloidal silica with particle size of 17 nm neutralized with ammonia: Levasil 200N30 marketed by AKZO Nobel.

### Example 11

Colorless varnish according to the present invention, prepared according to the general formula with 5% of 30% aqueous dispersion of colloidal silica with particle size of 17 nm neutralized with ammonia: Levasil 200N30 marketed by AKZO Nobel.

### Example 12

Colorless varnish according to the present invention, prepared according to the general formula with 1% of 30% aqueous dispersion of colloidal silica with particle size of 7 nm neutralized with ammonia: Ludox AS30 marketed by Grace.

### Example 13

Colorless varnish according to the present invention, prepared according to the general formula with 3% of 30% aqueous dispersion of colloidal silica with particle size of 7 nm neutralized with ammonia: Ludox AS30 marketed by Grace.

### Example 14

Colorless varnish according to the present invention, prepared according to the general formula with 5% of 40% aqueous dispersion of colloidal silica with particle size of 22 nm neutralized with soda: Ludox HS40 marketed by Grace.

### Example 15

Colorless varnish according to the present invention, prepared according to the general formula with 5% of 40% aqueous dispersion of colloidal silica with particle size of 12 nm neutralized with soda: Ludox TM40 marketed by Grace.

### Example 16

Colorless varnish according to the present invention, prepared according to the general formula with 5% of 40% aqueous dispersion of nonionic colloidal silica with particle size of 22 nm: Ludox TMA marketed by Grace.

### Example 17

Colored varnishes are prepared starting from the colorless varnishes of examples 1 to 16, and their stability at 20° C. and at 45° C. is observed. The various formulas tested are as follows:

Test Formula 1:

Colored varnish obtained by adding 3% of 30% dispersion of yellow organic pigment 1 to the colorless varnish of examples 1 to 3.

Test Formula 2:

Colored varnish obtained by adding 1% of 30% dispersion of red organic pigment 30 and 2% of nacre of granulometry between 20 and 160 micrometers to the colorless varnish of examples 1 to 15.

Test Formula 3:

Colored varnish obtained by adding 3% of 30% dispersion of titanium oxide to the colorless varnish of examples 1 to 15.

Test Formula 4:

Colored varnish obtained by adding 3% of 30% dispersion of yellow iron oxide to the colorless varnish of examples 1 to 3.

Test formula 5:

Colored varnish obtained by adding 3% of 30% dispersion of red iron oxide to the colorless varnish of examples 1 to 3.

Test formula 6:

Colored varnish obtained by adding 3% of 30% dispersion of blue ultramarine to the colorless varnish of examples 1 to 3.

Test formula 7:

Colored varnish obtained by adding 10% of silver 0.008 flakes (about 200 μm) to the colorless varnish of examples 1 to 6.

Results for Stability in Storage:

The results for stability after 6 months at 20° C. are presented in Table 2 below:

TABLE 2

| | | Example 2 | |
|---|---|---|---|
| | Example 1 | According to | Example 3 |
| | (comp.) | the invention | (comp.) |
| Test formula 1 | stable | stable | stable |
| Test formula 2 | sedimentation of the | stable | sedimentation of |
| | nacres | | the nacres |
| Test formula 3 | stable | stable | stable |
| Test formula 4 | slight sedimentation | stable | stable |
| Test formula 5 | slight sedimentation | stable | very slight |
| | | | sedimentation |
| Test formula 6 | slight sedimentation | stable | stable |
| Test formula 7 | sedimentation of the | stable | sedimentation of |
| | flakes | | the flakes |

The results for stability after 1 month at 45° C. are presented in Table 3 below:

TABLE 3

| | | Example 2 | |
|---|---|---|---|
| | Example 1 | According to | Example 3 |
| | (comp.) | the invention | (comp.) |
| Test formula 1 | stable | stable | stable |
| Test formula 2 | sedimentation of the | stable | sedimentation of |
| | nacres | | the nacres |
| Test formula 3 | slight sedimentation | stable | very slight |
| | | | sedimentation |
| Test formula 4 | sedimentation | stable | sedimentation |
| Test formula 5 | sedimentation | stable | sedimentation |
| Test formula 6 | sedimentation | stable | sedimentation |
| Test formula 7 | sedimentation of the | stable | sedimentation of |
| | flakes | | the flakes |

These results show that the best stability is obtained with the varnish composition according to the present invention containing the dispersion of colloidal silica.

Stability after 1 month at 45° C.: the effects of the concentration of colloidal silica dispersion and size of the silica particles were tested. The results are presented in Table 4.

TABLE 4

| | Example | | | | | | | |
|---|---|---|---|---|---|---|---|---|
| | 7 | 8 | 2 | 9 | 10 | 11 | 12 | 13 |
| % silica | 1 | 2 | 5 | 1 | 2 | 5 | 1 | 3 |
| dispersion | | | | | | | | |
| Particle | 22 | 22 | 22 | 17 | 17 | 17 | 7 | 7 |
| size (nm) | | | | | | | | |
| Counter-ion | NH₄⁺ | NH₄⁺ | NH₄⁺ | NH₄⁺ | NH₄⁺ | NH₄⁺ | NH₄⁺ | NH₄⁺ |
| Test | sedimentation | stable | stable | sedimentation | stable | stable | sedimentation | stable |
| formula 1 | | | | | | | | |
| Test | sedimentation | stable | stable | sedimentation | stable | stable | sedimentation | stable |
| formula 2 | | | | | | | | |
| Test | sedimentation | stable | stable | sedimentation | stable | stable | sedimentation | stable |
| formula 4 | | | | | | | | |

These results confirm that the minimum amount of colloidal silica dispersion must be above 1 wt % in the varnish composition.

Stability at 45° C.: the influence of the type of stabilization of the silica particles was observed, and is shown in Table 5 below.

TABLE 5

Example

| | 2 | 14 | 15 | 16 |
|---|---|---|---|---|
| % silica | 5 | 5 | 5 | 5 |
| dispersion | | | | |
| Particle | 22 | 22 | 12 | 22 |
| size nm | | | | |
| Counter-ion | NH₄⁺ | Na⁺ | Na⁺ | no counter-ion |
| Test formula 1 | stable | stable | stable | sedimentation |
| Test formula 2 | stable | stable | stable | sedimentation |

It appears that a dispersion of colloidal silica, stabilized ionically, preferably with sodium or ammonium ions, makes it possible to endow the polyurethane-based varnish composition with better stability.

1. A strippable aqueous nail varnish composition, comprising:
an aqueous polyurethane dispersion as film-forming agent, wherein it also comprises at least 1 wt % of an aqueous dispersion of anionic colloidal silica containing sodium or ammonium counter-ions and between 15 and 50 wt % of silica particles.

2. The composition as claimed in claim 1, wherein the aqueous dispersion of colloidal silica contains particles of colloidal silica of size between 5 and 45 nm.

3. The composition as claimed in either of claims 1, wherein the particle size of the colloidal silica is between 5 and 30 nm, more preferably between 7 and 25 nm.

4. The composition as claimed in claim 1, wherein the aqueous dispersion of colloidal silica contains between 30 and 40 wt % of silica particles.

5. The composition as claimed in claim 1, wherein said composition comprises a proportion of colloidal silica dispersion between 1 and 10 wt %, preferably between 2 and 5 wt %, relative to the total weight of the composition.

6. The composition as claimed in claim 1, wherein the polyurethane dispersion is an anionic dispersion, based on polyacrylate, polyether, polyester or polycarbonate and aliphatic and/or aromatic isocyanate, or a mixture thereof.

7. The composition as claimed in claim 1, wherein the polyurethane dispersion contains particles smaller than 200 nm, preferably smaller than 100 nm, and has a value of dry extract between 20 and 50 wt %, preferably between 20 and 40 wt %.

8. The composition as claimed in claim 1, wherein the polyurethane dispersion has a temperature of film formation less than or equal to 30° C., preferably below 20° C.

9. The composition as claimed in claim 1, wherein the composition preferably contains up to 30 wt % of an aqueous dispersion of acrylic or styrene acrylic polymer with particle size under 150 nm and preferably under 100 nm, as complementary film-forming agent.

10. The composition as claimed in claim 1, wherein the composition contains one or more organic or inorganic pigments and/or fillers in a proportion less than or equal to 10 wt %, preferably less than or equal to 5 wt %, relative to the total weight of the composition.

11. The composition as claimed in claim 1, wherein said composition also contains at least one additive selected from: co-solvents, coalescing agents, antifoaming agents, surfactants, wetting agents, dispersants, waxes, silicones, drying accelerators, crosslinking agents, adhesion promoters, UV filters, rheology agents such as associative acrylic and polyurethane thickeners, preservatives, such as antibacterial and antifungal agents necessary for preserving the aqueous medium, or a mixture thereof.

12. The composition as claimed in claim 1, wherein said composition contains one or more active agent(s) for the maintenance and aesthetic appearance of the nails, preferably with a nontherapeutic effect.

13. The composition as claimed in claim 1, wherein said composition is able to form a film on the nail which, after drying, can be removed by simple stripping.

14. A method for nail makeup and makeup removal, wherein said method comprises the following steps:
i) applying one or more layers of a varnish composition as claimed in claim 1, in the form of a continuous film, on the nail
ii) drying each of the layers of varnish at room temperature
iii) removing the varnish by peeling the film deposited on the nail.
